(19) United States
(12) Patent Application Publication
(10) Pub. No.: US 2024/0166776 A1
(43) Pub. Date: May 23, 2024

(54) USE OF PROTON-FORM ZEOLITE CATALYST TO PRODUCE LIGHT-POLYISOBUTYLENE

(71) Applicant: Braskem S.A., Camaçari (BR)

(72) Inventors: Richard Caulkins, Philadelphia, PA (US); Jiahan Xie, Philadelphia, PA (US); Alicia Brasselle, Philadelphia, PA (US); Richard Opfer, Philadelphia, PA (US); Dan Xie, Philadelphia, PA (US); Maria Giuliana Torraga Uchiyama, São Paulo City (BR); Marcio Henrique Dos Santos Andrade, São Paulo City (BR); João Capistrano Nobre De Abreu, São Paulo City (BR); Danniel Panza, São Paulo City (BR); Mauricio Silveira Cadore, São Paulo City (BR); Suzana Kupidlowski Boaventura, São Paulo City (BR); Jan Kalfus, Philadelphia, PA (US)

(73) Assignee: Braskem S.A., Camaçari (BR)

(21) Appl. No.: 18/517,947

(22) Filed: Nov. 22, 2023

Related U.S. Application Data
(60) Provisional application No. 63/427,607, filed on Nov. 23, 2022.

(51) Int. Cl. C08F 10/10; C08F 4/12

(52) U.S. Cl. CPC C08F 10/10; C08F 4/12; C08F 2410/06; C08F 2500/02; C08F 2500/17

(57) ABSTRACT

A method of forming a light-polyisobutylene may include polymerizing isobutene or an isobutene-containing monomer mixture in presence of a proton-form zeolite catalyst to form a light-polyisobutylene. A light-polyisobutylene may be formed by the method of polymerizing isobutene or an isobutene-containing monomer mixture in presence of a proton-form zeolite catalyst.

### BACKGROUND

Polyisobutylenes (PIB) are generally produced by cationic polymerization processes. Specifically, cationic polymerization is initiated by a proton donor species, by introducing a protic acid (Bronsted acid) or an aprotic acid (Lewis acid) with a proton donor. Protonic acids are species capable of donating protons, such as H⁺ ions, capable of interacting with the double bond present in the monomer and promoting the initiation of polymerization through the formation of the living polymeric chain. Cationic polymerization initiated through the use of a Lewis acid occurs in the presence of a proton donor, also known as an initiator or co-catalyst, such as: water, alcohol, organic acids or t-butyl chloride. The catalyst generates charged species from the co-catalyst to form the catalytic complex capable of initiating the polymerization of isobutylene with a proton-counterion pair.

Polyisobutylenes can be classified as light, low, medium, or heavy based on their molecular weight. Light-polyisobutylene has a molecular weight of 150-500 g/mol, preferentially with low polydispersity, low-molecular weight polyisobutylenes have a molecular weight of 500-5000 g/mol, medium molecular weight polyisobutylenes have a molecular weight of between 500 to 50000 g/mol, and high molecular weight polyisobutylenes have a molecular weight that is greater than 50,000 g/mol.

Currently, light-PIB is commonly produced as a by-product in the incumbent process to produce low molecular weight. Specifically, AlCl₃ catalyst, proton donor (water or HCl), and isobutylene or a hydrocarbon mixture containing isobutylene are co-fed into a reactor, and low molecular weight polyisobutylene is the major product. Through a separation process, a light-PIB component can be collected. The common disadvantages of such processes are: 1) low selectivity to light polyisobutylene as it is a by-product 2) the polydispersity is high; and 3) because AlCl₃ is used as a catalyst, there is residual chloride in the product, and additional energy and cost are needed to dissolve and neutralize the AlCl₃ catalyst.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect, embodiments disclosed herein relate to a method of forming a light-polyisobutylene that includes polymerizing isobutene or an isobutene-containing monomer mixture in presence of a proton-form zeolite catalyst to form a light-polyisobutylene.

In another aspect, embodiments disclosed herein relate to a light-polyisobutylene formed by the method of polymerizing isobutene or an isobutene-containing monomer mixture in presence of a proton-form zeolite catalyst.

Other aspects and advantages of the claimed subject matter will be apparent from the following description and the appended claims.

### BRIEF DESCRIPTION OF DRAWINGS

The present invention is described in connection with the attached FIGS. 1-6.

FIG. 1 shows the catalyst activity after 5 minutes at different temperatures.

FIG. 2 shows the catalyst activity after 5 minutes at different IB concentration.

FIG. 3 shows the catalyst activity as Arrhenius plots.

FIG. 4 shows the catalyst activity as Arrhenius plots, in view of IB concentration.

FIG. 5 shows the catalyst activity across the experiment duration.

FIG. 6 shows the product molecular weight Mₙ and polydispersity PD across the experiment duration.

### DETAILED DESCRIPTION

Embodiments of the present disclosure generally relate to a method of forming a light-polyisobutylene by polymerizing isobutene or an isobutene-containing monomer mixture in the presence of a proton-form zeolite catalyst. Use of a proton-form zeolite catalyst may advantageously allow for on purpose production of a light-polyisobutylene with a low polydispersity without halide residue.

Zeolite refers to a group of chemically related mineral crystalline, microporous aluminosilicate substances. A “proton-form zeolite” refers to a zeolite in which a proton balances the charge on framework aluminum atoms. The proton-form zeolite not only contains the protons to catalyze the cationic polymerization of isobutylene, but also has a microporous structure that can limit the size of the polyisobutylene product. These features make zeolite a suitable catalyst for on-purpose production of light polyisobutylene, as described herein.

### Light-Polyisobutylene

The light-polyisobutylene in accordance with one or more embodiments of the present disclosure may be represented by the formula (IB)ₙ where n is the number of monomer (IB) repeating units and ranges from 2 to 9. The number of repeating units may be a number having a lower limit of any of 2, 3, or 4, to an upper limit of any of 5, 6, 7, 8, or 9.

The light-polyisobutylene in accordance with one or more embodiments of the present disclosure may have a number average molecular weight (Mn) and/or a weight average molecular weight (Mw) ranging from about 100 g/mol to about 500 g/mol. The Mn and/or Mw may have a lower limit of any of 100 g/mol, 110 g/mol, 120 g/mol, 130 g/mol, 140 g/mol, 150 g/mol, 160 g/mol, 170 g/mol, 180 g/mol, 190 g/mol, 200 g/mol, 210 g/mol, 220 g/mol, 230 g/mol, 240 g/mol, 250 g/mol, 260 g/mol, 270 g/mol, 280 g/mol, 290 g/mol, 300 g/mol, 310 g/mol, 320 g/mol, 330 g/mol, 340 g/mol, or 350 g/mol, to an upper limit of any of 360 g/mol, 370 g/mol, 380 g/mol, 390 g/mol, 400 g/mol, 410 g/mol, 420 g/mol, 430 g/mol, 440 g/mol, 450 g/mol, 460 g/mol, 470 g/mol, 480 g/mol, 490 g/mol, or 500 g/mol. The molecular weight of the polyisobutylene product may be measured by gel permeation chromatography (GPC) by dissolving the polyisobutylene product in a suitable solvent.

The light-polyisobutylene in accordance with one or more embodiments of the present disclosure may have a polydispersity of less than 1.2. For example, the polydispersity may have a lower limit of 1.0 or 1.1.

The light-polyisobutylene in accordance with one or more embodiments of the present disclosure may have a kinematic viscosity ranging from about 3 to about 120 cSt at a specified temperature. The viscosity may have a lower limit of any of 3 cSt, 4 cSt, 5 cSt, 6 cSt, 10 cSt, 20 cSt, or 40 cSt, to an upper limit of any of 40 cSt, 60 cSt, 80 cSt, 105 cSt, 110 cSt, 115 cSt or 120 cSt. The viscosity mentioned above is measured at 37.8° C.

In a further embodiment, the light-polyisobutylene in accordance with the present disclosure may have a kinematic viscosity ranging from about 0.5 to about 120 cSt at a specified temperature. The viscosity may have a lower limit of any of 0.5 cSt, 1.0 cSt, 1.5 cSt, 2 cSt. 3 cSt, 4 cSt, 5 cSt, 6 cSt, 10 cSt, 20 cSt, or 40 cSt, to an upper limit of any of 40 cSt, 60 cSt, 80 cSt, 105 cSt, 110 cSt, 115 cSt or 120 cSt. The viscosity mentioned above is measured at 37.8° C.

### POLYMERIZATION OF ISOBUTYLENE

In one or more embodiments, the light-polyisobutylene is obtained through polymerization of isobutylene in a range of temperatures from −100° C. to about 150° C. For example, the polymerization may occur at a temperature range having a lower limit of any of −100° C., −75° C., −50° C., or −20° C. to an upper limit of any of 20° C., 40° C., 50° C., 60° C., 80° C., 100° C., 120° C., or 150° C., where any lower limit can be used in combination with any upper limit. For example, the polymerization may be performed at a temperature in between −20 ° C. and 50° C.to reduce the complexity and energy consumptions of the experimental procedure. When the polymerization is performed at or above the boiling temperature of the monomer or monomer mixture to be polymerized, it may be performed in pressure vessels, for example in autoclaves or in pressure reactors. In one or more embodiments, the polymerization may be carried out in a fixed-bed reactor, a fluidized bed reactor, a Micro-channel reactor, a continuous stirred-tank reactor a loop reactor, or a slurry reactor. In one or more embodiments, the reactor is pressurized at pressures in between ambient pressure and 50 bar.

In one or more embodiments of the present disclosure, the reaction time between the isobutylene monomer and the proton-form zeolite catalyst may be in the range of 1 to 100 minutes, for example from 5 to 25 minutes.

The polymerization reaction in accordance with one or more embodiments of the present disclosure may be quenched by separating the catalyst from the reaction medium.

Polymerizing isobutylene in the presence of the claimed catalyst may allow for the formation of light-polyisobutylene, i.e., a polyisobutylene with a molecular weight in range of between 100 to 500, directly without necessitating separation from a higher molecular weight fraction.

### Zeolite Catalyst

In one or more embodiments, isobutylene may be polymerized in the presence of a zeolite catalyst to form light-polyisobutylene. Zeolites have intrinsic acidity with at least some quantity of acid sites within the crystalline microporous aluminosilicate. The catalyst may be a proton-form zeolite, in which protons(s) balance the charge(s) of framework aluminum atoms to form acid sites. Zeolites in proton-form may have high acidity and high catalytic activity, and proton-exchanged zeolites with the degree of ion exchange higher than 50% may be even more suitable for use in the present disclosure. Activity of the proton-form zeolites may be dependent on the channel pore sizes of the zeolite, and in one or more embodiments, the proton-form zeolites may have a pore size of at least 4.85 angstrom and at most 100 angstrom. A pore size greater than 4.85 angstrom may accommodate the kinetic diameter of isobutylene thereby providing sufficient catalytic activity. A pore size less than 100 angstrom may provide sufficient steric hinderance to limit the size of produced polyisobutylene and provide sufficient Van der Waals interactions to enhance the catalytic activity of proton sites.

In one or more embodiments, the zeolites includes ferrierite (FER), mordenite (MOR), tschernichite (BEA), faujasite (FAU), or any other zeolite with pore >4.85 Å, including the frameworks AET, AFI, AFO, AFR, AFS, AFY, ATO, ATS, BEC, Boggsite (BOG), BOZ, BPH, CAN, CFI, CON, CSV, CTH, DFO, DON, EMT, EON, ETR, EUO, EZT, GME, GON, IFO, IFR, IFW, IMF, IRR, ISV, ITG, ITH, ITR, ITT, IWR, IWS, IWV, IWW, JSR, LTF, LTL, MAZ, MEI, MEL, MFS, MOZ, MSE, MTT, MTW, MWW, NES, OBW, OFF, OKO, OSI, OSO, PCS, POS, RWY, SAF, SAO, SBE, SBS, SBT, SEW, SFE, SFF, SFG, SFH, SFN, SFO, SFS, SOR, SOV, SSF, SSY, STF, STI, STW, SVY, SYT, TER, TON, TUN, UOV, USI, UTL, UWY, VET, VFI, and YFI.

Proton-form zeolites may be commercially obtained, or they may be produced from calcining ammonium-form zeolites. In one or more embodiments, the proton-form zeolite catalyst may be prepared by calcining commercial proton-form or ammonium-form zeolites in an inert environment. The calcining process may be performed in a range of temperatures from 400° C. to about 600° C. This calcination treatment is required even for zeolite catalysts which are already in proton-form in order to remove water which has been adsorbed by the zeolite from the atmosphere. In one or more embodiments of the present disclosure, the reaction time for the calcining step may be in the range of 4 to 18 hours. After the calcining step is complete, the catalyst should not be exposed to a water-containing atmosphere at temperatures less than 100° C. When exposed to water-free environments, the temperature may be reduced to the temperature of the polymerization reaction.

In one or more embodiments, the zeolite catalyst may have a catalytic activity calculated from the amount of isobutylene consumed, amount of catalyst, and reaction time using the equation below:

$$\\ Catalytic activity = \frac{moles of isobutylene consumed}{moles of catalyst * reaction time}$$

where catalytic activity is in mol_{IB}mol_{Al}⁻¹s⁻¹, moles of isobutylene consumed and moles of Al in moles, and reaction time in seconds.

In one or more embodiments, the catalytic activity may be greater than any of 0.02, 0.025, 003, 0.035, 0.04, 0.045, or 0.050 mol_{IB}mol_{Al}⁻¹s⁻¹ and less than 10 mol_{IB}mol_{Al}⁻¹s⁻¹.

### EXAMPLES

The following examples are merely illustrative and should not be interpreted as limiting the scope of the present disclosure.

### Example 1: Isobutylene Polymerization to Light Polyisobutylene in a Semi-Batch Reactor (at 25-35° C. and 10 psig Pressure)

### Materials

MFI, FER, BEA, and FAU zeolites were purchased from Zeolyst. Two different FAU zeolites were used: CBV720, a FAU zeolite with Si/Al 15 that is referred to here as FAU-15, and CBV760, a FAU zeolite with Si/Al 30 that is referred to here as FAU-30. Aluminum chloride (99.99%), Amberlyst® 16, Dried Amberlyst® 16, Amberlyst® 15H⁺, and dried Amberlyst® 15H⁺ were purchased from Sigma Aldrich. Hexane (>99%, anhydrous) was purchased from Sigma Aldrich. Isobutylene liquified gas cylinders were purchased from Air Gas (>99.5%, with less than 3 ppm water).

### Methods

The molecular weight of the light-polyisobutylene product was measured by Gel Permeation Chromatography (GPC) using a Gel Permeation Chromatography and Size Exclusion Chromatography (SEC) system (Infinity 1260) with a refractive index detector and Oligopore column from Agilent Technologies. The column was calibrated using polystyrene standards.

For some samples, the molecular weight of the light-polyisobutylene was also measured by gas chromatography (GC). In these cases, the molecular composition of the light-polyisobutylene product was determined using an Agilent 8890 Gas Chromatograph with a flame ionization detector (FID) and a DB-FastFAME column. The GC-based number-average molecular weight (Mₙ), weight-average molecular weight (M_{w}), and polydispersity (PD) were calculated from the molecular composition according to the formulas below, where ni is the molar composition of species i and Mᵢ is the molecular weight of species i:

$$\\ {M}_{n} = \frac{\sum {n}_{i} {M}_{i}}{\sum {n}_{i}}$$

$$\\ {M}_{w} = \frac{\sum {n}_{i} {M}_{i}^{{}_{} 2}}{\sum {n}_{i} {M}_{i}}$$

$$\\ PD = \frac{{M}_{w}}{{M}_{n}} .$$

The activity of the catalyst was determined using the formula below, where catalytic activity is in mol_{IB}mol_{Al}⁻¹s⁻¹, moles of isobutylene consumed and moles of Al in moles, and reaction time in seconds:

$$\\ Catalytic activity = \frac{moles of isobutylene consumed}{moles of Aluminum * reaction time}$$

### Preparation of Mesoporous FAU Zeolite Catalysts

In order to demonstrate the role of channel size on the formation of light polyisobutylene, FAU-15 and FAU-30 were modified by a NaOH treatment to add mesopores in the 20-70 Å range. The modified samples are designated as FAU-15-1, FAU-15-2, FAU-30-1, and FAU-30-2. To prepare these samples, 6 grams each of FAU-15 and FAU-30 powders were mixed with 40 mL of NaOH solution at varying concentrations. FAU-15-1 and FAU-30-1 were prepared using 0.2N NaOH solutions, while FAU-15-2 and FAU-30-2 were prepared using 0.4N NaOH solutions. The mixtures were stirred at room temperature for 30 minutes and then hydrothermally treated at 80° C. for 12 hours. The resulting solid products were separated from the solution through filtration, followed by washing with deionized water.

Afterward, the products were treated with 60 mL of 1N ammonium nitrate solution at 95° C. for 2 hours. The solution was cooled and decanted; this process was repeated three times. Subsequently, the powders were dried at 90° C. before undergoing calcination in a muffle furnace in the presence of air, heated to 400° C. at a rate of 2° C./min, held at 400° C. for five hours, and finally cooled to ambient temperature.

X-ray diffraction (XRD) was performed for each of these materials to confirm that they retained the FAU structure. The acidity of these materials was characterized by ammonia temperature programmed desorption (NH₃-TPD). Brunauer-Emmett-Teller (BET) analysis was also performed to obtain the pore size distribution of these samples.

### Preparation of the Proton-Form Zeolite Catalyst Composition

In the following example, proton-form zeolites catalyst compositions were prepared by calcining MFI, BEA, MOR, and Y-type zeolites at 500-550° C. for 4-5 hours. The prepared proton-form zeolite catalysts were then cooled to 100-150° C. and transferred to a glove box. Then, the MFI, BEA, MOR, and Y-type zeolites were placed in individual reactors. Table 1 shows the different catalysts used. Comparative Example 1 is a catalyst commonly used to produce PIB commercially.

TABLE 1

| | | | | | Acid site | % of Pore |
|---|---|---|---|---|---|---|
| | | | | Channel | count | Volume in |
| | | | | pore | (μmol | 20-70 Å |
| Sample | | Catalyst | Si/Al | size | H g⁻¹) | mesopores |
| 1 | (CE) | AlCl₃ + water | NA | NA | NA | NA |
| 2 | (I) | Amberlyst ® 16 | NA | NA | NA | NA |
| 3 | (I) | Dried | NA | NA | NA | NA |
| | | Amberlyst ® 16 | | | | |
| 4 | (I) | Amberlyst ® 15 H⁺ | NA | NA | NA | NA |
| 5 | (I) | Dried Amberlyst | NA | NA | NA | NA |
| | | 15 H⁺ | | | | |
| 6 | (I) | Amberlite ® CG50 | NA | NA | NA | NA |
| 7 | (I) | Dried Amberlite ® | NA | NA | NA | NA |
| | | CG50 | | | | |
| 8 | (I) | MFI (ZSM5) | 15 | <4.84 Å | NA | NA |
| 9 | (I) | FER | 10 | <4.84 Å | NA | NA |
| 10 | (I) | BEA | 19 | >4.84 Å | NA | NA |
| 11 | (I) | FAU-15 (Y) | 15 | >4.84 Å | 584 | 5.5 |
| 12 | (I) | FAU-30 (Y) | 30 | >4.84 Å | 453 | 7.3 |
| 13 | (I) | FAU-15-1 (Y) | NA | >4.84 Å | 495 | 12.8 |
| 14 | (I) | FAU-15-2 (Y) | NA | >4.84 Å | 411 | 46.6 |
| 15 | (I) | FAU-30-1 (Y) | NA | >4.84 Å | 202 | 34.6 |
| 16 | (I) | FAU-30-2 (Y) | NA | >4.84 Å | 105 | 60.3 |

### Polymerization of Isobutylene to Light-Polyisobutylene

The reactors with the catalysts were removed from the glove box. The amount of Al in each reactor was constant at 0.05 mmol for examples 1-11. Due to the low Al content of FAU-30 materials, the mass of catalyst was instead held constant at 50 mg for examples 12-16. 50 mg corresponds to 0.05 mmol Al for FAU-15 (example 11). Pure isobutylene gas was added into the reactor under vigorous stirring at 600 rpm and the total pressure was maintained at 10 psig for 30 mins at 25-35° C. (298-308K). The isobutylene input was closed after 30 minutes, and the reactor was depressurized. A 0.2 um PTFE filter was used to remove the catalyst from the reaction medium. The reaction medium was analyzed directly by GC-FID for examples 11-16 before any additional treatment was conducted. Following GC analysis (if conducted), the unreacted isobutylene and solvent were removed by evaporation at 80° C. (353 K) overnight. The amount of polyisobutylene formed was measured using a precise balance with 0.1 mg deviation. Product composition was then measured using GPC.

Table 2 below shows the catalytic activity, molecular weight (Mn and Mw), and polydispersity (PD) of the different catalyst compositions.

TABLE 2

| | | | Activity | Mₙ | Mw | | Mₙ | |
|---|---|---|---|---|---|---|---|---|
| | | | (mol_{IB} | (g/mol) | (g/mol) | | (g/mol) | PD |
| Sample | | Catalyst | mol_{Al}⁻¹ s⁻¹) | (GPC) | (GPC) | PD (GPC) | (GC) | (GC) |
| 1 | (CE) | AlCl₃ + water | 0.05 | 309 | 400 | 1.29 | NA | NA |
| 2 | (I) | Amberlyst ® | <0.005 | NA | NA | NA | NA | NA |
| | | 16 | | | | | | |
| 3 | (I) | Dried | <0.005 | NA | NA | NA | NA | NA |
| | | Amberlyst ® | | | | | | |
| | | 16 | | | | | | |
| 4 | (I) | Amberlyst ® | <0.005 | NA | NA | NA | NA | NA |
| | | 15 H⁺ | | | | | | |
| 5 | (I) | Dried | <0.005 | NA | NA | NA | NA | NA |
| | | Amberlyst ® | | | | | | |
| | | 15 H⁺ | | | | | | |
| 6 | (I) | Amberlite ® | <0.005 | NA | NA | NA | NA | NA |
| | | CG50 | | | | | | |
| 7 | (I) | Dried | <0.005 | NA | NA | NA | NA | NA |
| | | Amberlite ® | | | | | | |
| | | CG50 | | | | | | |
| 8 | (I) | MFI (ZSM5) | <0.005 | NA | NA | NA | NA | NA |
| 9 | (I) | FER | 0.020 | NA | NA | NA | NA | NA |
| 10 | (I) | BEA | 0.045 | 226 | 234 | 1.01 | NA | NA |
| 11 | (I) | FAU-15 (Y) | 0.041 | 223 | 226 | 1.01 | 198 | 1.07 |
| 12 | (I) | FAU-30 (Y) | 0.094 | 246 | 257 | 1.05 | 164 | 1.12 |
| 13 | (I) | FAU-15-1 (Y) | 0.094^{a} | 248 | 258 | 1.04 | 163 | 1.13 |
| 14 | (I) | FAU-15-2 (Y) | 0.154^{a} | 264 | 281 | 1.06 | 162 | 1.13 |
| 15 | (I) | FAU-30-1 (Y) | 0.289^{a} | 240 | 248 | 1.03 | 164 | 1.11 |
| 16 | (I) | FAU-30-2 (Y) | 0.361^{a} | NA | NA | NA | 147 | 1.12 |

^{a}Al content of the modified FAU catalysts may be affected by the mesoporosity treatment. Rates are instead normalized for these samples to the acid site content as determined by NH₃-TPD. It is assumed that one acid site exists per Al.

As seen in Table 2, aluminum chloride catalyst (comparative example 1) produces a low molecular weight polyisobutylene with a molecular weight of 309 g/mol. however the polyisobutylene produced has a high polydispersity at 1.29. Non-zeolite solid acid catalysts (inventive samples 2-7) exhibit negligible reactivity of under 0.005 mol_{IB}mol_{Al}⁻¹s⁻¹.

Interestingly, the activity of proton-form zeolite catalysts is highly dependent on the channel size. Specifically, zeolites with a pore size less than the kinetic diameter of isobutylene, such as MFI and FER, exhibit poor activity (inventive samples 8 and 9) of under 0.005 mol_{IB}mol_{Al}⁻¹s⁻¹.

Proton-type BEA and FAU-15 (inventive samples 10 and 11) exhibit similar activity to incumbent AlCl₃ catalyst normalized by total Al of 0.045 and 0.041 mol_{IB}mol_{A1}⁻¹s⁻¹ respectively. BEA and FAU-15 produce light-PIB with molecular weight (Mn) of 226 and 223 g/mol respectively (isobutylene tetramer) with a narrow polydispersity of about 1.01.

For FAU catalysts, molecular weight was characterized by GC as well as GPC. This was done to better quantify formation of diisobutylene, which is a potential component of light-polyisobutylene which lies outside the calibration range of GPC standards and may evaporate under the 80° C. overnight treatment. Proton-form FAU-15 gives a GC-based Mₙ of 198 g/mol compared to a GPC-based Mn of 223 g/mol, which is the result of 15% weight selectivity to diisobutylene. In contrast, proton-form FAU-30 (inventive sample 12) has a GC-based Mₙ of 164 g/mol and a GPC-based Mₙ of 246 g/mol; this difference is the result of 45% mass selectivity to the diisobutylene component of the light-polyisobutylene product.

Mesoporous FAU-15 and FAU-30 (inventive samples 13-16) demonstrate the role of pore size in controlling product selectivity. The composition of the light-polyisobutylene product becomes enriched in diisobutylene with the addition of mesopores, with the GC-based Mₙ decreasing from 198 g/mol for unmodified FAU-15 to 162 g/mol for mesoporous FAU-15-2. Similarly, the GC-based Mₙ decreased from 164 g/mol for unmodified FAU-30 to 147 g/mol for mesoporous FAU-30-2. The molecular weight (and therefore viscosity) of the light polyisobutylene obtained from a proton-form zeolite catalyst may therefore be altered through modifications to the pore size. Interestingly, following the 80° C. evaporation treatment, all samples produced light-polyisobutylene with molecular weight >220 g/mol and polydispersity <1.07, indicating that the viscosity of the final light-polyisobutylene product can be tuned by first separating out low molecular weight components such as diisobutylene.

Each disclosed catalyst not only produced light-polyisobutylene with a low polydispersity index, but also is in solid phase and therefore is easier to store, handle and remove from the product reducing the cost of production. Furthermore, it is envisioned the catalyst can be easily reused, resulting in a greener process that reduces the cost of the catalyst.

### Example 2: Isobutylene Polymerization to Light Polyisobutylene in a Batch Reactor (at 0-60° C. and at a Range of Isobutylene Concentrations)

### Materials

Hexane (98.5%, mixture of isomers) was purchased from Sigma Aldrich. Nitrogen (>99.999%) and isobutylene liquified gas (>99.5%, with less than 3 ppm water) were purchased from Airgas. 3A molecular sieves and quartz wool (fine, 4 μm) were purchased from Thermo Fisher Scientific. FAU (Y-type) zeolite with Si/Al 15 was purchased from Zeolyst. The 3A molecular sieves were used to remove any water present in the hexane before use.

### Methods

The molecular composition of the light-polyisobutylene product was determined using an Agilent 8890 Gas Chromatograph (GC) with a flame ionization detector (FID) and a DB-FastFAME column. The number-average molecular weight (Mₙ), weight-average molecular weight (M_{w}), and polydispersity (PD) were calculated from the molecular composition according to the formulas below, where nᵢ is the molar composition of species i and Mᵢ is the molecular weight of species i:

$$\\ {M}_{n} = \frac{\sum {n}_{i} {M}_{i}}{\sum {n}_{i}}$$

$$\\ {M}_{w} = \frac{\sum {n}_{i} {M}_{i}^{{}_{} 2}}{\sum {n}_{i} {M}_{i}}$$

$$\\ PD = \frac{{M}_{w}}{{M}_{n}}$$

The activity of the catalyst was determined in units of (mol isobutylene)*(mol Al)⁻¹*s⁻¹ using the formula below:

$$\\ Activity = \frac{moles of PIB produced}{( moles of aluminum ) * ( reaction time )} .$$

### Preparation of the Proton-Form Zeolite Composition

FAU (Y-type) zeolite was pelletized, crushed, and sieved to a particle size between 106-150 μm. The proton-form zeolite catalyst composition was prepared by calcining the sieved catalyst at 500° C. for 4 hours. The proton-form zeolite catalyst was then cooled to 150° C. and transferred into a nitrogen environment inside a glovebox until it was used.

### Polymerization of Isobutylene to Light-Polyisobutylene

Catalyst activity was assessed in a stainless-steel batch reactor. For each experiment, a mass of catalyst corresponding to 0.1 mmol Al was loaded into the reactor inside a glovebox. Catalyst was loaded into a compartment built into the reactor lid and the compartment was sealed with a cap. The reactor was removed from the glovebox and nitrogen was used to purge any air present in the reactor before adding 100 mL hexane to the reactor. Pure gaseous isobutylene was added at 0° C. under vigorous stirring by an impeller at 650 rpm. Sufficient isobutylene was added to achieve the target isobutylene concentration, between 1.2-3.6 mol L⁻¹. The amount of isobutylene added was measured with 0.1 mg accuracy using a balance to give a precise calculation of the isobutylene concentration. Following isobutylene addition, the reactor was heated to the target reaction temperature, between 0-60° C.

When the target temperature was reached, nitrogen was used to pressurize the catalyst compartment, resulting in the release of catalyst into the reaction mixture and pressurizing the reactor to 100 psig. The nitrogen input was closed, and the reaction was allowed to proceed for 40 minutes. The reaction mixture was sampled at 5-, 10-, 20-, and 40-minute intervals. A 0.2 μm PTFE filter was used to remove any catalyst present in the samples as they were collected. Samples were analyzed by GC-FID.

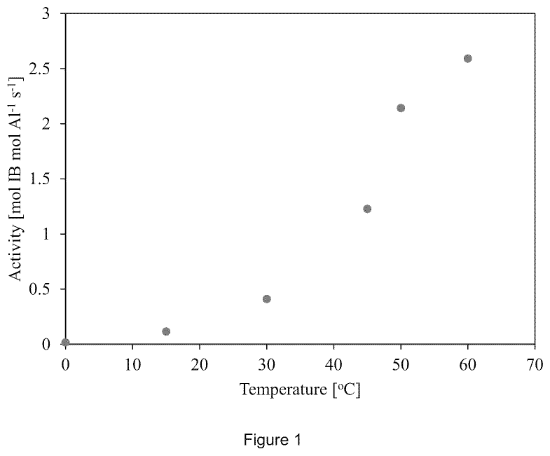

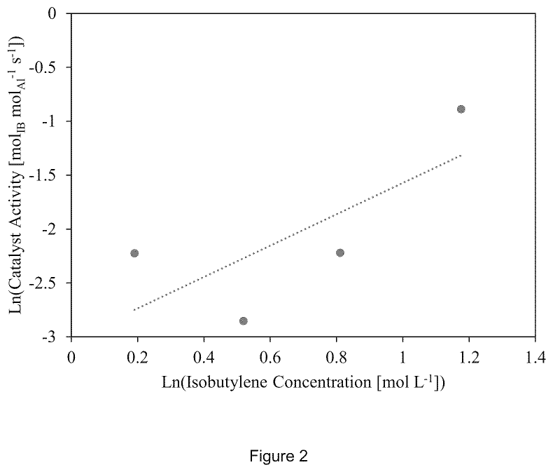

Catalyst activity was assessed at a range of temperatures from 0-60° C. and an isobutylene concentration between 3.2-3.6 mol L⁻¹. Activity after 5 minutes at each temperature is shown in FIG. 1, and the corresponding molecular weight Mₙ and polydispersity PD are shown in FIG. 2. The catalyst activity was above 0.05 molyB mol_{Al}⁻¹ s⁻¹ across the range 15-60° C. The molecular weight Mₙ of the products was above 119 g mol⁻¹ across this range, with a polydispersity PD less than 1.12.

Table 3 below shows the isobutylene concentration, molecular weight number (Mn), and polydispersity (PD) at different temperatures within the defined range.

TABLE 3

| Temperature | Isobutylene Concentration | Mn (g mol⁻¹) | PD |
|---|---|---|---|
| (° C.) | (mol L⁻¹) | (GC) | (GC) |
| 0 | 3.61 | 112 | 1.00 |
| 15 | 3.58 | 126 | 1.06 |
| 30 | 3.24 | 133 | 1.11 |
| 45 | 3.40 | 122 | 1.06 |
| 50 | 3.41 | 122 | 1.05 |
| 60 | 3.33 | 119 | 1.04 |

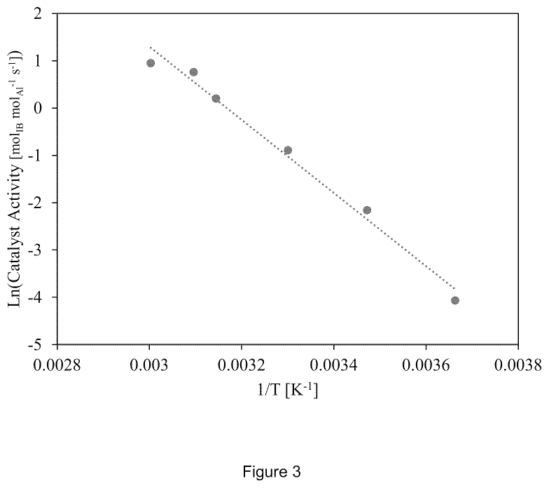

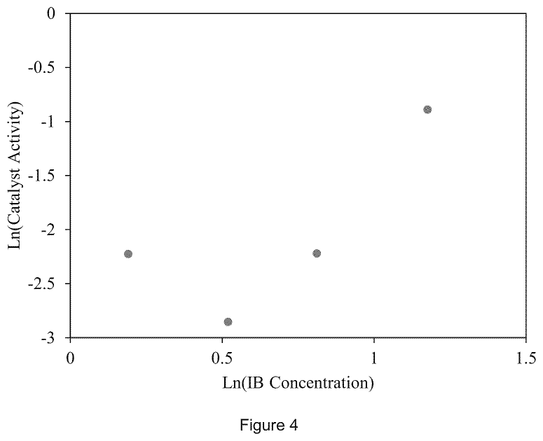

Catalyst activity was similarly assessed at a range of isobutylene concentrations from 1.2-3.6 mol L⁻¹ while holding temperature constant at 30° C. Activity after 5 minutes at each concentration can be seen in FIG. 3, and the corresponding molecular weight Mₙ and polydispersity PD can be seen in FIG. 4. The catalyst exhibited good activity above 0.035 mol_{IB}mol_{Al}⁻¹ s⁻¹ across the entire range 1.2-3.6 mol L⁻¹. The molecular weight was above 120 g mol⁻¹ across this range, indicating the formation of light polyisobutylene. The polydispersity also remained narrow, remaining below 1.12 at all conditions.

Table 4 below shows the isobutylene concentration, molecular weight number (Mn), and polydispersity (PD) at different temperatures within the defined range.

TABLE 4

| Temperature | Isobutylene Concentration | Mn (g mol⁻¹) | PD |
|---|---|---|---|
| (° C.) | (mol L⁻¹) | (GC) | (GC) |
| 30 | 1.21 | 136 | 1.13 |
| 30 | 1.68 | 120 | 1.05 |
| 30 | 2.25 | 131 | 1.12 |
| 30 | 3.24 | 133 | 1.11 |

The data from these experiments of Example 2 can be used to fit the parameters to an Arrhenius equation:

$$\\ Rate = A * exp ( - \frac{{E}_{app}}{RT} ) * {C}_{IB}^{{}_{} n}$$

Wherein rate refers to the catalytic activity for light polyisobutylene formation, A is a pre-exponential factor, Eₐₚₚ is the apparent activation energy, R is the ideal gas constant, T is the reaction temperature, C_{IB} is the isobutylene concentration, and n is the reaction order. Fitting the data as Arrhenius plots in FIG. 3 and FIG. 4 yields an apparent activation energy of 56.8 KJ/mol and a reaction order of 2.13, as shown in Table 5, below.

TABLE 4

| Apparent Activation Energy (kJ mol⁻¹) | Reaction order |
|---|---|
| 56.8 | 2.13 |

### Example 3: Isobutylene Polymerization to Light Polyisobutylene in a Fixed-Bed Flow Reactor

### Materials

Hexane (98.5%, mixture of isomers) was purchased from Sigma Aldrich. Nitrogen (>99.999%) and isobutylene liquified gas (>99.5%, with less than 3 ppm water, with 150 psi helium pad) were purchased from Airgas. 3A molecular sieves (2-5 mm beads) and quartz wool (fine, 4 μm) were purchased from Thermo Fisher Scientific. FAU (Y-type) zeolite with Si/Al 15 was purchased from Zeolyst. The 3A molecular sieves were used to remove any water present in the hexane before use.

### Methods

The molecular composition of the light-polyisobutylene product was determined using an Agilent 8890 Gas Chromatograph (GC) with a flame ionization detector (FID) and a DB-FastFAME column. The number-average molecular weight (Mₙ), weight-average molecular weight (M_{w}), and polydispersity (PD) were calculated from the molecular composition according to the formulas below, where ni is the molar composition of species i and Mᵢ is the molecular weight of species i:

$$\\ {M}_{n} = \frac{\sum {n}_{i} {M}_{i}}{\sum {n}_{i}}$$

$$\\ {M}_{w} = \frac{\sum {n}_{i} {M}_{i}^{{}_{} 2}}{\sum {n}_{i} {M}_{i}}$$

$$\\ PD = \frac{{M}_{w}}{{M}_{n}}$$

The activity of the catalyst was determined in units of (mol PIB)*(mol Al)⁻¹*s⁻¹ using the formula below:

$$\\ Activity = \frac{( molar flow rate of PIB produced )}{( moles of aluminum )}$$

### Preparation of the Proton-Form Zeolite Composition

FAU (Y-type) zeolite was pelletized, crushed, and sieved to a particle size between 106-150 μm. The sieved catalyst was calcined at 500° C. for 4 hours, then cooled to 150° C. and transferred into a nitrogen environment inside a glovebox until used.

### Polymerization of Isobutylene to Light-Polyisobutylene

Catalyst activity was assessed in a fixed-bed flow reactor. This reactor consisted of a length of 316 stainless steel tubing with outer diameter ⅜″. An amount of catalyst sufficient to provide 0.1 mmol Al was loaded into the reactor inside the glovebox. The catalyst bed was secured in place between thin layers of quartz wool. The reactor was sealed against exposure to atmosphere by closed valves, removed from the glovebox, and installed into the experimental apparatus. The system was heated to 30° C. and pressurized to 70 psig under 20 sccm flowing nitrogen. Nitrogen flow was stopped, and 2.5 mL/min hexane and 0.5 mL/min isobutylene were pumped through the reactor for 5.5 hours. The reactor outlet stream was sampled at 15-minute intervals for the first two hours and at 30-minute intervals for the final 3.5 hours, and liquid samples were analyzed by GC-FID.

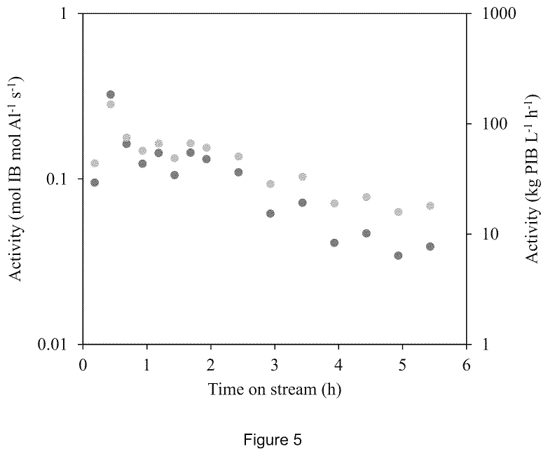

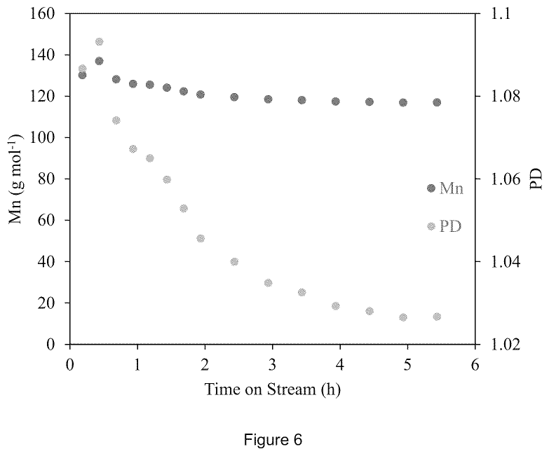

The catalyst activity for the experiment duration is shown in FIG. 5, and the product molecular weight Mₙ and polydispersity PD are shown in FIG. 6. Catalyst activity exceeded 0.03 mol_{IB}mol_{Al}s⁻¹ for the duration of the experiment. This reaction rate exceeds the 1 kg L⁻¹h⁻¹ commonly cited as the minimum rate required for industrial application. Mₙ remained above 117 g mol⁻¹ and PD remained below 1.1 for the duration of the experiment.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims. In the claims, means-plus-function clauses are intended to cover the structures described herein as performing the recited function and not only structural equivalents, but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures. It is the express intention of the applicant not to invoke 35 U.S.C. § 112(f) for any limitations of any of the claims herein, except for those in which the claim expressly uses the words ‘means for’ together with an associated function.

What is claimed:

1. A method of forming a light-polyisobutylene, comprising:
polymerizing isobutene or an isobutene-containing monomer mixture in presence of a proton-form zeolite catalyst to form a light-polyisobutylene.

2. The method of claim 1, wherein the light-polyisobutylene has a viscosity of 3-120 cst at 37.8° C.

3. The method of claim 1, wherein the light-polyisobutylene has a viscosity of 0.5-120 cst at 37.8° C.

4. The method of claim 1, wherein the light-polyisobutylene has a formula (IB)ₙ where n ranges from 2 to 9 and has a molecular weight (Mn) ranging from 110-500 g/mol.

5. The method of claim 4, wherein n ranges from 4 to 8.

6. The method of claim 1, wherein the light-polyisobutylene has a polydispersity index of less than 1.2.

7. The method of claim 1, wherein the proton-form zeolite catalyst is a crystalline microporous aluminosilicate.

8. The method of claim 7, wherein the microporous aluminosilicate has a pore size greater than 4.85 angstrom.

9. The method of claim 1, further comprising:
calcining a zeolite catalyst to form the proton-form zeolite catalyst.

10. The method of claim 9, wherein the zeolite catalyst comprises ferrierite (FER), mordenite (MOR), tschernichite (BEA), faujasite (FAU) or any other zeolite with pore size greater than 4.85 Angstrom.

11. The method of claim 10, wherein the zeolite catalyst has a framework selected from the group consisting of AET, AFI, AFO, AFR, AFS, AFY, ATO, ATS, BEC, Boggsite (BOG), BOZ, BPH, CAN, CFI, CON, CSV, CTH, DFO, DON, EMT, EON, ETR, EUO, EZT, GME, GON, IFO, IFR, IFW, IMF, IRR, ISV, ITG, ITH, ITR, ITT, IWR, IWS, IWV, IWW, JSR, LTF, LTL, MAZ, MEI, MEL, MFS, MOZ, MSE, MTT, MTW, MWW, NES, OBW, OFF, OKO, OSI, OSO, PCS, POS, RWY, SAF, SAO, SBE, SBS, SBT, SEW, SFE, SFF, SFG, SFH, SFN, SFO, SFS, SOR, SOV, SSF, SSY, STF, STI, STW, SVY, SYT, TER, TON, TUN, UOV, USI, UTL, UWY, VET, VFI, and YFI.

12. The method of claim 9, wherein the calcining occurs at a temperature ranging between 400 and 600° C.

13. The method of claim 9, wherein the calcining occurs for a time ranging between 4 and 18 h.

14. A light-polyisobutylene formed by the method of claim 1.
